(19) United States
(12) Patent Application Publication
(10) Pub. No.: US 2020/0009478 A1
(43) Pub. Date: Jan. 9, 2020

(54) FERMENTATION REACTOR AND FERMENTATION PROCESS

(71) Applicant: UNIBIO A/S, Odense (DK)

(72) Inventors: Budi Juliman Hidayat, Hvidovre (DK); Ib Christensen, Allerod (DK); Patrick Nicklas Pedersen, Tollose (DK); Subir Kumar Nandy, Kgs. Lyngby (DK); Henrik Busch-Larsen, Odense M (DK)

(21) Appl. No.: 16/490,120

(22) PCT Filed: Feb. 28, 2018
(86) PCT No.: PCT/EP2018/054944

§ 371 Date: Aug. 30, 2019

(30) Foreign Application Priority Data
Mar. 1, 2017 (DK) PA 2017 00144

(51) Int. Cl. B01D 19/00; C12M 1/00; C12M 1/34; C12M 1/36

(52) U.S. Cl. CPC B01D 19/0063; C12M 29/18; C12M 41/48; C12M 41/32; C12M 29/20

(57) ABSTRACT

A fermentation reactor with a loop-part includes a circulation pump, a degassing-tank, and a fermentation liquid outlet for withdrawing fermentation liquid from the fermentation reactor, the degassing-tank including: (i) a first outlet connecting the degassing-tank to a first leg of the loop-part and allowing fermentation liquid present in the degassing-tank to flow into the loop-part; (ii) a first inlet connecting the degassing-tank to a second leg of the loop-part, allowing fermentation liquid present in the loop-part to flow into the degassing-tank; and (iii) a vent tube for discharging effluent gasses from the degassing tank, such as CO₂; wherein one or more of the following criteria applies: (a) the loop-part is provided with an anti-vortex device; (b) certain volumetric ratios pertain between volumes relative to cross sections of reactor parts; (d) ratios pertain between different cross sectional areas of reactor parts.

### TECHNICAL FIELD OF THE INVENTION

The present invention relates to a fermentation reactor and a fermentation process for cultivating an organism. In particular, the present invention relates to a fermentation reactor suitable for improved cultivation of methanotrophic bacterial cells providing a single cell protein.

### BACKGROUND OF THE INVENTION

Loop-reactors comprises circulation pumps having propeller blades designed for pumping a mixture of liquid and gas. The gases are introduced at different locations throughout the loop-part, but typically they will be supplied at the upper end of the downstream part of the loop-part. By introducing the gases at the upper end of the downstream part of the loop a nearly non-compressed injection may be obtained, since the gases only have to overcome a hydrostatic pressure of some few meters. It is desirable to provide a fine dispersion of the gases in the fermentation medium as the gas to liquid mass transfer rate will be improved. A fine dispersion of the gases in the liquid is traditionally affected by means of passive or inactive static mixing elements placed immediately below the gas injectors. The liquid flow in the downstream part of the loop must be sufficiently high so that all the injected gas is carried together with the flow and down through the static mixers. Here a comminution of the gas is affected so that a large number of small gas bubbles is obtained, which are dispersed uniformly in the liquid. The bubbles are carried along with the liquid flow down through the downstream part of the loop to its lower end and further on through a horizontal part to the vertical part of the loop so that the gas bubbles are re-dispersed (e.g. by means of static mixing elements) several times in the liquid.

Most prior art describes that the in-line circulation pump is placed in the horizontal part or close to the horizontal part of the fermentation reactor because it is then considered to assist in producing a re-dispersion of the gas in the liquid and partially because it is practical to have it placed at the bottom of the fermenter.

However, gas bubbles in liquids have a tendency to fuse together to larger volumes (coalesce). This tendency is undesirable as it makes the gas to liquid mass transfer less effective which has a negative consequence on productivity of the biomass. Coalescence of the gas in liquid may also be affected by a reduction in the hydrostatic pressure. This, reduction in hydrostatic pressure may be counteracted providing a pressure in the loop-part.

WO 2010/069313 describes a fermenter and a method of fermentation in a U-shape and/or nozzle U-loop fermenter comprising a U-part having an essentially vertical down-flow part, an essentially vertical up-flow part and a substantially horizontal connecting part, which connects the lower ends of the down-flow part and the up-flow part, a top part which is provided above the U-part and has a diameter which is substantially larger than the diameter of U-part, means for creating liquid circulation in the U-part of the fermenter, and one or more gas injection points for the introduction and dispersion of the gas(ses) into the fermentation liquid. The pressure may be controlled in the loop-part by placing the circulation pump in the upper part of the down-flow part and a valve in the upper part of the up-flow part, resulting in creation of a pressurized zone between the circulation pump and the valve.

Hence, an improved fermentation reactor and process would be advantageous, and in particular a more efficient, more stable and reliable process resulting in higher productivity would be advantageous.

### SUMMARY OF THE INVENTION

Thus, an object of the present invention relates to a fermentation reactor suitable for improved cultivation of organisms, in particular, methanotrophic bacterial cells, providing a single cell protein.

In particular, it is an object of the present invention to provide a fermentation reactor and a process for cultivating an organism that solves the above-mentioned problems of the prior art and is more efficient, more stable and reliable and resulting in higher productivity.

Accordingly, one aspect of the invention relates to a fermentation reactor comprising a loop-part having a circulation pump, a degassing-tank, and a fermentation liquid outlet for withdrawing fermentation liquid from the fermentation reactor, said degassing-tank comprises:
(i) a first outlet connecting the degassing-tank to a first leg of the loop-part and allowing fermentation liquid present in the degassing-tank to flow into the loop-part;
(ii) a first inlet connecting the degassing-tank to a second leg of the loop-part, allowing fermentation liquid present in the loop-part to flow into the degassing-tank; and
(iii) a vent tube for discharging effluent gasses from the degassing tank, such as CO₂;
   wherein one or more of the following criteria applies;
(a) wherein the first outlet and/or the first leg of the loop-part is provided with an anti-vortex device to avoid flooding of the circulation pump;
(b) wherein a ratio between a volume (internal volume determined as m³) of the degassing-tank relative to a volume (internal volume determined as m³) of the loop-part is in the range of 0.25:1 to 20:1; e.g. in the range of 0.5:1 to 15:1; such as in the range of 1:1 to 12:1; e.g. in the range of 2:1 to 10:1; such as in the range of 3:1 to 8:1; e.g. in the range of 4:1 to 6:1;
(c) wherein a numerical ratio between a volume (internal volume, determined as m³) of the degassing-tank relative to a cross section area of the first leg and/or the cross section area (determined as m²) of second leg is in the range of 10:1 to 250:1; such as in the range of 20:1 to 200:1, e.g. in the range of 30:1 to 150:1, such as in the range of 40:1 to 100:1, e.g. about 50:1;
(d) wherein a cross section area of the first leg (determined as m²) is 10% or more larger than a cross section area of the second leg (determined as m²), such as 20% larger or more; e.g. 30% larger or more, such as 40% larger or more; e.g. 50% larger or more, such as 60% larger or more; e.g. 70% larger or more, such as 80% larger or more; e.g. 90% larger or more, such as 100% larger or more; e.g. 125% larger or more, such as 150% larger or more; e.g. 200% larger or more.
(e) wherein a cross section area of the second leg (determined as m²) is 10% or more larger than a cross section area of the first leg (determined as m²), such as 20% larger or more; e.g. 30% larger or more, such as 40% larger or more; e.g. 50% larger or more, such as 60% larger or more; e.g. 70% larger or more, such as 80% larger or more; e.g. 90% larger or more, such as 100% larger or more; e.g. 125% larger or more, such as 150% larger or more; e.g. 200% larger or more; or
any combination of criteria (a) to (e).

Another aspect of the present invention relates to a fermentation process for production of biomass by cultivating an organism, the method comprises the steps of:
(1) supplying a fermentation medium to the fermentation reactor;
(2) adding the organism to a fermentation reactor according to the present invention;
(3) allowing the organism present in the fermentation medium to ferment, providing a fermentation liquid; and
(4) recovering the fermentation liquid from the fermentation reactor providing the biomass.

### DETAILED DESCRIPTION OF THE INVENTION

The inventors of the present invention surprisingly found that by introducing different physical features of the construction of a loop reactor relative to the publicly available loop reactor, results in a more efficient, stable and reliable process providing a higher productivity of biomass.

A preferred embodiment of the present invention relates to a fermentation reactor comprising a loop-part having a circulation pump, a degassing-tank, and a fermentation liquid outlet for withdrawing fermentation liquid from the fermentation reactor, said degassing-tank comprises:
(i) a first outlet connecting the degassing-tank to a first leg of the loop-part and allowing fermentation liquid present in the degassing-tank to flow into the loop-part;
(ii) a first inlet connecting the degassing-tank to a second leg of the loop-part, allowing fermentation liquid present in the loop-part to flow into the degassing-tank; and
(iii) a vent tube for discharging effluent gasses from the degassing tank, such as CO₂;
   wherein one or more of the following criteria applies;
(a) wherein the first outlet and/or the first leg of the loop-part is provided with an anti-vortex device to avoid flooding of the circulation pump;
(b) wherein a ratio between a volume (internal volume determined as m³) of the degassing-tank relative to a volume (internal volume determined as m³) of the loop-part is in the range of 0.25:1 to 20:1; e.g. in the range of 0.5:1 to 15:1; such as in the range of 1:1 to 12:1; e.g. in the range of 2:1 to 10:1; such as in the range of 3:1 to 8:1; e.g. in the range of 4:1 to 6:1;
(c) wherein a numerical ratio between a volume (internal volume, determined as m³) of the degassing-tank relative to a cross section area of the first leg and/or the cross section area (determined as m²) of second leg is in the range of 10:1 to 250:1; such as in the range of 20:1 to 200:1, e.g. in the range of 30:1 to 150:1, such as in the range of 40:1 to 100:1, e.g. about 50:1;
(d) wherein a cross section area of the first leg (determined as m²) is 10% or more larger than a cross section area of the second leg (determined as m²), such as 20% larger or more; e.g. 30% larger or more, such as 40% larger or more; e.g. 50% larger or more, such as 60% larger or more; e.g. 70% larger or more, such as 80% larger or more; e.g. 90% larger or more, such as 100% larger or more; e.g. 125% larger or more, such as 150% larger or more; e.g. 200% larger or more.
(e) wherein a cross section area of the second leg (determined as m²) is 10% or more larger than a cross section area of the first leg (determined as m²), such as 20% larger or more; e.g. 30% larger or more, such as 40% larger or more; e.g. 50% larger or more, such as 60% larger or more; e.g. 70% larger or more, such as 80% larger or more; e.g. 90% larger or more, such as 100% larger or more; e.g. 125% larger or more, such as 150% larger or more; e.g. 200% larger or more.
(f) wherein the loop-part of the fermentation reactor comprises one or more active devices for distributing gas in the fermentation liquid;
(g) wherein a volume (internal volume determined as m³) of the degassing-tank is 25% or more of the volume (internal volume determined as m³) of the loop-part, e.g. 50% or more, such as 100% or more, e.g. 200% or more, such as 300% or more, e.g. 400% or more, such as 300% or more, e.g. 500% or more, such as 600% or more, e.g. 700% or more, such as 800% or more, e.g. 900% or more;
(h) wherein a numerical volume (internal volume, determined in m³) of the degassing-tank is at least 20 times larger than a cross section area (determined in m²) of the first leg and/or a cross section area (determined as m²) of the second leg, such as at least 30 times larger, e.g. at least 50 times larger, such as at least 75 times larger, e.g. at least 100 times larger, such as at least 125 times larger, e.g. at least 150 times larger, such as at least 200 times larger;
(i) wherein a numerical volume (internal volume, determined in m³) of the loop-part relative to the numerical length (determined in meters) of the loop-part is in the range of 0.01-10, such as in the range of 0.05-8, e.g. in the range of 0.1-6, such as in the range of 0.5-4, e.g. in the range of 1-2;
(j) wherein a numerical volume (internal volume, determined in m³) of the loop-part is at least 2 times smaller than the numerical length (determined in meters) of the loop-part, such as at least 5 times smaller, e.g. at least 10 times smaller, such as at least 25 times smaller, e.g. at least 50 times smaller, such as at least 75 times smaller, e.g. at least 100 times smaller, such as at least 150 times smaller, e.g. at least 200 times smaller;
(k) wherein the ratio between the length (determined in meters) of a horizontal part of the loop-part and the length (determined in meters) of a vertical part is in the range of 0.5:1 to 1:20; in the range of 1:1 to 1:15; such as in the range of 1:2 to 1:12, e.g. in the range of 1:3 to 1:10, such as in the range of 1:5 to 1:8; and/or
(l) wherein a numerical ratio between a volume (internal volume determined in m³) of the degassing-tank relative to a cross section area (determined as m²) of the first inlet or the first outlet is in the range of 10:1 to 200:1; such as in the range of 20:1 to 100:1, e.g. in the range of 30:1 to 80:1; such as in the range of 40:1 to 60:1, e.g. about 50:1.
(m) wherein a numerical volume (internal volume, determined in m³) of the degassing-tank is at least 20 times larger than a numerical cross section area (determined as m²) of the first inlet and/or a cross section area (determined as m²) of the first outlet, such as at least 30 times larger, e.g. at least 50 times larger, such as at least 75 times larger, e.g. at least 100 times larger, such as at least 125 times larger, e.g. at least 150 times larger; or any combination of criteria (a) to (m).

In particular, the A preferred embodiment of the present invention relates to a fermentation reactor comprising a loop-part having a circulation pump, a degassing-tank, and a fermentation liquid outlet for withdrawing fermentation liquid from the fermentation reactor, said degassing-tank comprises:
(iv) a first outlet connecting the degassing-tank to a first leg of the loop-part and allowing fermentation liquid present in the degassing-tank to flow into the loop-part;
(v) a first inlet connecting the degassing-tank to a second leg of the loop-part, allowing fermentation liquid present in the loop-part to flow into the degassing-tank; and
(vi) a vent tube for discharging effluent gasses from the degassing tank, such as CO₂;
   wherein one or more of the following criteria applies;
(a) wherein the first outlet and/or the first leg of the loop-part is provided with an anti-vortex device to avoid flooding of the circulation pump;
(b) wherein a ratio between a volume (internal volume determined as m³) of the degassing-tank relative to a volume (internal volume determined as m³) of the loop-part is in the range of 0.25:1 to 20:1; e.g. in the range of 0.5:1 to 15:1; such as in the range of 1:1 to 12:1; e.g. in the range of 2:1 to 10:1; such as in the range of 3:1 to 8:1; e.g. in the range of 4:1 to 6:1;
(c) wherein a numerical ratio between a volume (internal volume, determined as m³) of the degassing-tank relative to a cross section area of the first leg and/or the cross section area (determined as m²) of second leg is in the range of 10:1 to 250:1; such as in the range of 20:1 to 200:1, e.g. in the range of 30:1 to 150:1, such as in the range of 40:1 to 100:1, e.g. about 50:1;
(d) wherein a cross section area of the first leg (determined as m²) is 10% or more larger than a cross section area of the second leg (determined as m²), such as 20% larger or more; e.g. 30% larger or more, such as 40% larger or more;

e.g. 50% larger or more, such as 60% larger or more; e.g. 70% larger or more, such as 80% larger or more; e.g. 90% larger or more, such as 100% larger or more; e.g. 125% larger or more, such as 150% larger or more; e.g. 200% larger or more.
(e) wherein a cross section area of the second leg (determined as m²) is 10% or more larger than a cross section area of the first leg (determined as m²), such as 20% larger or more; e.g. 30% larger or more, such as 40% larger or more; e.g. 50% larger or more, such as 60% larger or more; e.g. 70% larger or more, such as 80% larger or more; e.g. 90% larger or more, such as 100% larger or more; e.g. 125% larger or more, such as 150% larger or more; e.g. 200% larger or more; or
   any combination of criteria (a) to (e).

In the context of the present invention, the term “internal volume” relates to the open cavity inside a tank or inside a pipe encircled by the walls of the pipe of the tank.

The above criteria provide an optimized fermentation tank comprising a loop-part, which has been optimized considering practical work with the fermentation tank; stability; efficiency; reliability and costs (both production costs and establishing costs).

In an embodiment of the present invention at least two of the above criteria applies, such as at least 3 of the criteria applies, e.g. at least 4 of the criteria applies, such as at least 5 of the criteria applies, e.g. at least 6 of the criteria applies, such as at least 7 of the criteria applies, e.g. at least 8 of the criteria applies, such as at least 9 of the criteria applies, e.g. at least 10 of the criteria applies.

In a further embodiment of the present invention at least criteria (a) applies. Preferably, criteria (a) applies in combination with one or more of criteria (b)-(m). In an even further embodiment of the present invention at least criteria (f) applies. Preferably, criteria (f) applies in combination with one or more of criteria (a)-(e) or (g)-(m). In yet an embodiment of the present invention, criteria (a) and/or criteria (f) applies in combination with one or more of criteria (b)-(e) and/or (g)-(m) applies, such as in combination with two or more of criteria (b)-(e) and/or (g)-(m) applies, e.g. in combination with 3 or more of criteria (b)-(e) and/or (g)-(m) applies, such as in combination with 4 or more of criteria (b)-(e) and/or (g)-(m) applies, e.g. in combination with 5 or more of criteria (b)-(e) and/or (g)-(m) applies, such as in combination with 6 or more of criteria (b)-(e) and/or (g)-(m) applies, e.g. in combination with 7 or more of criteria (b)-(e) and/or (g)-(m) applies.

In an embodiment of the present invention the degassing tank may preferably be a top-tank, placed on top of the loop-part. Preferably the length (meters, m) of the degassing tank is larger than the diameter (meters, m) of the degassing tank.

In a further embodiment of the present invention the degassing tank may be a cylindrically shaped or prismatic shaped tank. Preferably, the prismatic shaped degassing-tank has triangular end faces, square end faces, pentagonal end faces, or hexagonal end faces.

Depending on the design of the fermentation reactor the fermentation liquid outlet may be placed various places. In an embodiment of the present invention the fermentation liquid outlet for withdrawing fermentation liquid from the fermentation reactor is placed in the degassing-tank and/or in the loop-part.

In the context of the present invention, the term “circulation pump” relates to a pump placed in the loop-part and responsible for driving the fermentation liquid around in the fermentation reactor, from the degassing-tank through the loop-part and back to the degassing-tank. The term “circulation pump” relates to a pump pumping the fermentation liquid in a substantially parallel direction relative to the flow direction of fermentation liquid in the loop-part.

In an embodiment of the present invention the circulation pump according to the present invention may be placed in the upper half part of the first leg. Preferably, the circulation pump may be placed in the first leg close to the first outlet. Even more preferably, the circulation pump may be placed adjacent to and downstream from an anti-vortex device.

In a further embodiment of the present invention the circulation pump may be a propeller pump, a lobe pump or a turbine pump.

The fermentation reactor may preferably comprise means for providing an increased pressure in the fermentation reactor. The benefit from subjecting the fermentation liquid to an increase is that the gas to liquid mass transfer rate of the substrate gases will increase, and productivity and/or effectivity will increase.

By placing the recirculation pump in the upper half of the first leg the gas to liquid mass transfer rate may even further benefit from the hydrostatic pressure generated as the fermentation liquid moves from the degassing-tank through the first outlet into to the first leg and through the circulation pump, which may be placed in the upper half of the first leg as described herein, and the further down the first leg where the hydrostatic pressure keeps increasing as the fermentation liquid moves down.

In order to generate an increased pressure in the loop-part, a counter-pressure, against the increased flow, e.g. provided by the circulation pump, needs to be provided. Thus, in an embodiment of the present invention a flow reducing device may be inserted. Preferably, the flow reducing device may be placed upstream from the first inlet and in the second leg.

In an embodiment of the present invention the flow reducing device may be an adjustable flow reducing device.

In a further embodiment of the present invention, the flow reducing device may be an active flow reducing device.

Preferably, the flow reducing device may be a valve, a hydrocyclone, a pump, or a propeller pump, preferably a valve.

In the context of the present invention the flow reducing device is not an inactive or a static device, such as a static mixer.

In an embodiment of the present invention the fermentation reactor according to the present invention may comprise one or more gas injection points. Preferably, the one or more gas injection points may be followed by one or more active devices for distributing gas in the fermentation process and/or one or more inactive mixing members. During operation, gases may be introduced into the loop-part, as described earlier, in order to be able to introduce the gases into the fermentation reactor during operation the pressure of the gas may be around 2 to 3 bar above the pressure inside the U-loop. Hence due to the hydrostatic pressure generated the higher in the loop-part the gases are introduced the lower the pressure needs to be over the gas injector pressure.

The one or more inactive mixing members may be a static mixer which static mixers may preferably be immediately following the one or more gas injection points in order to assist comminuting the gas bubbles, and/or avoid or limit coalescence of the gas bubbles, in the fermentation liquid.

Once inside the fermentation reactor and the loop-part, the gas bubbles may be subjected to two competing processes, bubble break up and bubble coalescence. The final average bubble size will be determined by the more dominant, or faster, process of the two. If bubble coalescence is very slow compared to bubble breakup, the average bubble diameter is determined by the breakup process, however, if the bubbles coming out of the gas inlet are smaller than the maximum stable bubble diameter, the average bubble diameter is determined by the bubble formation process. On the other hand, if coalescence occurs rapidly, bubbles coming out of the gas inlet may coalesce and grow larger until they exceed the maximum stable bubble size, after which bubble break up occurs. Since gas bubble break up depends on the local velocities of eddies/local turbulences, there are local coalescence-break up equilibria taking place, resulting in a variation of bubble size throughout the loop-part.

In an embodiment of the present invention the fermentation reactor may comprise an ion sensor or analyzer for determining the content of one or more ion species in a fermentation liquid. Preferably, the one or more ion species may be selected from phosphate, calcium, hydrogen, nitrate, and/or ammonium, preferably nitrate.

In a further embodiment of the present invention the fermentation reactor; the one or more sensors or analyzers; the one or more gas inlet; the one or more water inlet; the one or more fermentation medium inlet is coupled to a computer.

Preferably, the one or more gas inlet; the one or more water inlet; the one or more fermentation medium inlet is controlled to a computer based on the data obtained from the one or more sensors or analyzers.

The fermentation reactor may further comprise at least one water inlet. The water supplied through the at least one water inlet may be tap water or demineralized water, preferably demineralized water to avoid the components present in tap water to influence on the composition of the fermentation medium.

In an embodiment of the present invention the fermentation reactor may comprise at least one fermentation medium inlet.

The length of the loop-part of the fermentation reactor may influence the productivity of the fermentation reactor and the fermentation process as the organism absorbs nutritional gasses introduced and growth, as biomass generation is conducted while traveling through the loop-part of the fermenter. During the biomass generation and growth of the organism, waste gases like CO₂ may be produced. These waste gases produced, e.g. CO₂ needs to be transported to the degassing-tank, where the waste gases may be released and discharged from the fermentation reactor.

Hence the loop-part of the fermentation reactor may have a length that is sufficient to allow sufficient growth of the biomass and/or sufficient utilization of the nutritional gases added, however, the length for the loop-part should not be so long that growth would be inhibited due to a to high content of waste gases, e.g. CO₂, in the fermentation liquid.

In an embodiment of the present invention the loop-part of the fermentation reactor may have a length of at least 10 meters, such as at least 20 meters, e.g. at least 30 meters, such as at least 40 meters, e.g. at least 50 meters, such as at least 75 meters, e.g. at least 100 meters, such as at least 125 meters, e.g. at least 150 meters.

In the present context, the length of the loop-part of the fermentation reactor may be determined based on the centerline of the loop-part.

The present invention relates to a fermentation reactor as described in item (i) to (iii) and optionally elsewhere in this description wherein the loop-part of the fermentation reactor comprises one or more active devices for distributing gas in the fermentation liquid.

In an embodiment of the present invention the one or more active devices for distributing gas in the fermentation liquid may be a micronanosparger for introducing and/or distributing gas into the fermentation liquid; and/or a dynamic motion device, such as a dynamic mixer.

Micronanospargers, may be provided in the fermentation reactor according to the present invention to create very small bubbles injected into the loop-part. This active (instead of passive) way to create bubbles may thus provide the organism inside the loop-part with bubbles of smaller size which are otherwise non-existent should only a sparger is used. The gas bubbles created using a micronanosparger would have a diameter of less than 50 microns, such as less than 40 microns, e.g. less than 30 microns, such as less than 20 microns, e.g. less than 15 microns.

As an alternative or as a supplement to the micronanosparger a dynamic motion device may be provided. The dynamic motion device may be triggered by a direct or indirect supply of energy, i.e. the use of dynamic mixer, instead of static mixer. The dynamic mixer according to the present invention may be intended to specifically enhance the gas/liquid mixing. The use of dynamic mixer allows more energy imparted into the culture inside the U-loop reactor in the most optimal way for achieving better gas/liquid mixing.

In an embodiment of the present invention the dynamic mixer may be in the first leg and/or in the second leg of the loop-part. This placement of the dynamic mixers provides the possibility to enhance the mixing even further by exploiting the proximity of the dynamic mixer to the inner wall of the loop-part. For example, the gas bubbles coming out of the dynamic mixer can be directed outward, instead of parallel with the flow path of the fermentation liquid, along the leg, to hit the inner wall of the loop-part. The extra eddies caused by the collision between the gas bubbles in the fermentation liquid and the inner wall of the leg is believed to further increase the mixing and decrease the bubble size.

Either directly or indirectly driven mixer may be used.

In a further embodiment of the present invention the one or more active devices for distributing gas in the fermentation liquid may be placed in-line in the loop-part of the fermentation reactor and can enhance mixing of gas and fermentation liquid.

In a preferred embodiment of the present invention, the one or more active devices for distributing gas in the fermentation liquid may be placed in-line in the loop-part of the fermenter and placed in a non-parallel angle relative to the flow of the fermentation liquid.

The one or more active devices for distributing gas in the fermentation liquid may be placed in-line in the loop-part of the fermenter at, or close to, the bottom part of the first leg; the bottom part of the second leg; and/or the horizontal part of the loop-part.

When e.g. the circulating pump may be located in the upper part of the first leg as described above and close to the first outlet of the degassing-tank there may be a risk of vortex. Vortex may occur when pump speed gets high for a given surface level. The disadvantages of vortex may be that gas may be brought to the vicinity of the pump, resulting in cavitation with reduced pump efficiency and damaged pump blades or casing as a follow up effect.

The present invention relates to a fermentation reactor as described in item (i) to (iii) and optionally elsewhere in this description wherein the first outlet and/or the first leg of the loop-part is provided with an anti-vortex device to avoid flooding of the circulation pump.

The anti-vortex device may be installed in the degassing-tank; in the first outlet, or in the first leg.

The anti-vortex device may be a device formed and/or installed to break the flow and/or channelling of the vortex.

The present invention relates to a fermentation reactor as described in item (i) to (iii) and optionally elsewhere in this description wherein:
a ratio between a volume (internal volume determined as m³) of the degassing-tank relative to a volume (internal volume determined as m³) of the loop-part is in the range of 0.25:1 to 20:1; e.g. in the range of 0.5:1 to 15:1; such as in the range of 1:1 to 12:1; e.g. in the range of 2:1 to 10:1; such as in the range of 3:1 to 8:1; e.g. in the range of 4:1 to 6:1;
a volume (internal volume determined as m³) of the degassing-tank is 25% or more of the volume (internal volume determined as m³) of the loop-part, e.g. 50% or more, such as 100% or more, e.g. 200% or more, such as 300% or more, e.g. 400% or more, such as 300% or more, e.g. 500% or more, such as 600% or more, e.g. 700% or more, such as 800% or more, e.g. 900% or more; and/or
a volume (internal volume determined as m³) of the degassing-tank is in the range of 25%-1500% of the volume (internal volume determined as m³) of the loop-part, e.g. 50%-1200%, such as 100%-1000%, e.g. 200%-800%, such as 400%-600%.

The advantage of this ratio between the volume (internal volume determined as m³) of the degassing-tank relative to the volume (internal volume determined as m³) of the loop-part may be that an improved fed-batch fermentation process; an improved degassing in the degassing-tank may be obtained.

The present invention relates to a fermentation reactor as described in item (i) to (iii) and optionally elsewhere in this description wherein:
a numerical ratio between a volume (internal volume, determined as m³) of the degassing-tank relative to a cross section area of the first leg and/or the cross section area (determined as m²) of second leg is in the range of 10:1 to 250:1; such as in the range of 20:1 to 200:1, e.g. in the range of 30:1 to 150:1, such as in the range of 40:1 to 100:1, e.g. about 50:1; and/or
a numerical volume (internal volume, determined in m³) of the degassing-tank is at least 20 times larger than a cross section area (determined in m²) of the first leg and/or a cross section area (determined as m²) of the second leg, such as at least 30 times larger, e.g. at least 50 times larger, such as at least 75 times larger, e.g. at least 100 times larger, such as at least 125 times larger, e.g. at least 150 times larger, such as at least 200 times larger;

The advantage of this ratio between the numerical volume (internal volume, determined in m³) of the degassing-tank relative to the cross section area (determined in m²) of the first leg and/or the cross section area (determined as m²) of the second leg, may be that less fountain may be observed and a reduced risk of gas being brought to the vicinity of the pump and a reduced risk of cavitation may be obtained.

In the present context, the term “fountain” relates to the splashes of fermentation liquid when entering the degassing-tank via the first inlet.

In the present context, the term “cavitation” relates to the formation of vapor or gas cavities in a liquid (i.e. small liquid-free zones like bubbles). This may be the consequence of forces acting upon the liquid. “cavitation” may be a significant cause of wear in some engineering contexts. Collapsing bubbles that implode near to a metal surface cause cyclic stress through repeated implosion. This results in surface fatigue of the metal causing the type of wear called “cavitation”.

The present invention relates to a fermentation reactor as described in item (i) to (iii) and optionally elsewhere in this description wherein:
a numerical volume (internal volume, determined in m³) of the loop-part relative to the numerical length (determined in meters) of the loop-part is in the range of 0.01-10, such as in the range of 0.05-8, e.g. in the range of 0.1-6, such as in the range of 0.5-4, e.g. in the range of 1-2;
a numerical volume (internal volume, determined in m³) of the loop-part is at least 2 times smaller than the numerical length (determined in meters) of the loop-part, such as at least 5 times smaller, e.g. at least 10 times smaller, such as at least 25 times smaller, e.g. at least 50 times smaller, such as at least 75 times smaller, e.g. at least 100 times smaller, such as at least 150 times smaller, e.g. at least 200 times smaller;

The advantage of this ratio between the numerical volume (internal volume, determined in 20 m³) of the loop-part relative to the numerical length (determined in meters) of the loop-part may be that improved mixing of the fermentation liquid and the nutrient gases; improved productivity; longer retention time may be obtained.

The present invention relates to a fermentation reactor as described in item (i) to (iii) and optionally elsewhere in this description wherein:
the ratio between the length (determined in meters) of a horizontal part of the loop-part and the length (determined in meters) of a vertical part is in the range of 0.5:1 to 1:20; in the range of 1:1 to 1:15; such as in the range of 1:2 to 1:12, e.g. in the range of 1:3 to 1:10, such as in the range of 1:5 to 1:8;

The advantage of this ratio between the length (determined in meters) of a horizontal part of the loop-part relative to the length (determined in meters) of a vertical part may be that a sufficient increase in gas solubility due to the hydrostatic pressure may be obtained and/or a possible loss of gases from the liquid to the possibly present empty space above the liquid flowing in the horizontal part of the U-loop may be omitted.

A preferred embodiment of the present invention relates to a fermentation process for production of biomass by cultivating an organism, the method comprises the step of:
(1) supplying a fermentation medium to the fermentation reactor;
(2) adding the organism to a fermentation reactor according to the present invention;
(3) allowing the organism present in the fermentation medium to ferment, providing a fermentation liquid; and
(4) recovering the fermentation liquid from the fermentation reactor providing the biomass.

In an embodiment of the present invention the organism is at least one methanotrophic organism, or a combination of organisms comprising at least one methanotrophic organism.

Preferably, the microorganism is a bacterium or a yeast. Preferably, the bacteria comprise at least one methanotrophic bacteria, or a combination of organisms comprising at least one methanotrophic bacteria.

In an embodiment of the present invention the methanotrophic bacteria is selected from a Methylococcus. Preferably, Methylococcus capsulatus.

In order to ensure sufficient minerals and/or ions in the fermentation liquid one or more ion species of the fermentation liquid may be analyzed during the fermentation process. Preferably, the one or more ion species is selected from phosphate, calcium, hydrogen, nitrate, and/or ammonium.

In an embodiment of the present invention a sensor or analyzer may be used to analyze the one or more ion species, preferably, the sensor or analyzer is an in-line sensor or an in-line analyzer.

In a further embodiment of the present invention, the one or more sensors or analyzers; the one or more gas inlet; the one or more water inlet; the one or more fermentation medium inlet may be controlled by a computer.

In a further embodiment of the present invention, the one or more gas inlet; the one or more water inlet; the one or more fermentation medium inlet may be controlled and/or operated by a computer based on the data obtained from the one or more sensors or analyzers.

The fermentation process according to the present invention may be a batch fermentation, a fed-batch fermentation or a continuous fermentation process. Preferably the fermentation process is a continuous fermentation process (a production process).

In an embodiment of the present invention the pressure inside the loop-part and in the bottom part of the loop-part may be in the range of 0 to 6 bar g, such as in the range of 1-5.5 bar g, e.g. in the range of 2-5 bar g, such as in the range of 2.5-4.5 bar g, e.g. in the range of 3-4 bar g.

In the context of the present invention, the term “bar g” relates to a pressure above the ambient pressure of 1 bar. Hence, 0 bar g is the same as a total pressure of 1 bar, 1 bar g is the same as a total pressure of 2 bar etc.

In a further embodiment of the present invention the pressure inside the loop-part and in the upper part of the first leg and downstream from the circulation pump may be in the range of 0 to 6 bar g above ambient, such as in the range of 1-5.5 bar g, e.g. in the range of 2-5 bar g, such as in the range of 2.5-4.5 bar g, e.g. in the range of 3-4 bar g.

In another embodiment of the present invention the pressure inside the degassing-tank may be in the range 0 - 1 bar g, such as in the range of 0.1-0.75 bar g, e.g. in the range of 0.2-0.5 bar g.

In an embodiment of the present invention the fermentation reactor may comprise the one or more of the above criteria in combination with the basic fermentation reactor as described in WO 2010/069313, and/or as described in WO 2000/70014, which both are hereby incorporated by reference.

Examples of fermentation reactor design; use of sensors and analysers; and controlling of a fermentation reactor has been described in WO 2010/069313; and/or WO 2000/70014, which both are hereby incorporated by reference.

The present invention will now be described in more detail in the following non-limiting preferred embodiment of the present invention.

### PREFERRED EMBODIMENT OF THE INVENTION

The present invention relates to a fermentation reactor comprising a loop-part having a circulation pump, a degassing-tank, and a fermentation liquid outlet for withdrawing fermentation liquid from the fermentation reactor, said degassing-tank comprises a first outlet connecting the degassing-tank to a first leg of the loop-part and allowing fermentation liquid present in the degassing-tank to flow into the loop-part. The fermentation reactor further comprises a first inlet connecting the degassing-tank to a second leg of the loop-part, allowing fermentation liquid present in the loop-part to flow into the degassing-tank and a vent tube for discharging effluent gasses from the degassing tank, such as CO₂. The first leg has an upper half where the circulation pump may be placed. The second leg has an upper half comprising a flow reducing device. The area between the circulation pump, downstream from said circulation pump, and the flow reducing device, upstream from said circulation pump, may comprise an increased pressure relative to the pressure in the degassing tank.

In order to improve the gas to liquid mass transfer and hence improve productivity of the fermentation process one or more active devices for distributing gas in the fermentation liquid may be introduced into the loop-part. Preferably, one or more active devices for distributing gas in the fermentation liquid may be placed in the horizontal part of the loop-part. One or more active devices for distributing gas in the fermentation liquid may also be placed in the vertical part of the loop-part, preferably in the first leg, but some active devices for distributing gas in the fermentation liquid may also be placed in the vertical second leg. The fermentation reactor may also comprise an anti-vortex device to avoid flooding of the circulation pump. This anti-vortex device may be places in the first outlet or between the first outlet and the circulation pump. The anti-vortex device may be provided to avoid air from being sucked from the degassing tank to the circulating pump which would seriously affect the productivity of the process and in worst case destroy the circulation pump.

The fermentation reactor may be shaped in many different ways; in one embodiment of the present invention the fermentation reactor comprises a vertical part which is significantly longer than the horizontal part, preferably the vertical part is at least 5 times longer than the horizontal part. The benefit of having a longer vertical part than horizontal part is that the closer to the bottom of the loop-part the fermentation liquid comes the higher the pressure, hydrostatic pressure, the fermentation liquid will be subjected to. When the pressure increases down through the first leg of the fermentation reactor the gas to liquid mass transfer rate will increase. Even further advantages of utilizing this hydrostatic pressure may be provided if the circulation pump is placed in the upper half of the first leg. Further information on this and additional features for controlling and providing a pressure in the loop part may also be suitable in the present invention and has been described in WO 2010/069313, which is hereby incorporated by reference. The same phenomenon applies when the fermentation liquid moves from the horizontal part up through the second leg where the hydrostatic pressure decreases and the gas to liquid mass transfer rate will decrease and change to a liquid to gas mass transfer rate of CO₂ excreted from the organism and the following degassing in the degassing tank may become easier. In an alternative embodiment of the present invention the shape of the fermentation reactor may comprise a horizontal part which is significantly longer than the vertical part, preferably the horizontal part is at least 5 times longer than the vertical part.

During operation, the fermentation reactor may be provided with an organism, preferably a bacterial cell, in particular a methanotrophic bacterial cell capable of converting methane into biomass. When continuous fermentation of the organism is started, a fermentation liquid may be obtained from the fermentation liquid outlet for withdrawing fermentation liquid from the fermentation reactor, while fermentation medium is simultaneously added to the fermentation reactor, via a fermentation medium inlet. Feed gas supply members; water supply members; acid/base supply members and/or fermentation medium supply members may be provided to allow continuous supply of feed gases, water and fermentation medium during the fermentation process (including the initial batch fermentation, the following fed-batch fermentation, and the continuous fermentation). Preferably, the fermentation reactor comprises one or more sensors/analyzers for determining the concentration of at least one of the ion species, phosphate, ammonium, nitrate and hydrogen ion; a gas sensor/analyzer for determining the gas concentration (e.g. methane and/or oxygen concentration), and at least one temperature sensor/analyzer. The sensors/analyzers may preferably deliver signals to a data processing system (a computer), wherein the signals received are processed and the dosage of feed gases, water, minerals and acid/bases for pH adjustment via the supply members are calculated and optimized from pre-programmed amounts relative to the results measured. Further details on the fermentation process, the sensors/analyzers and controlling of the process using a computer is described in WO 00/70014, which is hereby incorporated by reference.

It should be noted that embodiments and features described in the context of one of the aspects of the present invention also apply to the other aspects of the invention.

All patent and non-patent references cited in the present application, are hereby incorporated by reference in their entirety.

1. A fermentation reactor comprising a loop-part having a circulation pump, a degassing-tank, and a fermentation liquid outlet for withdrawing fermentation liquid from the fermentation reactor, said degassing-tank comprises:
(i) a first outlet connecting the degassing-tank to a first leg of the loop-part and allowing fermentation liquid present in the degassing-tank to flow into the loop-part;
(ii) a first inlet connecting the degassing-tank to a second leg of the loop-part, allowing fermentation liquid present in the loop-part to flow into the degassing-tank; and
(iii) a vent tube for discharging effluent gasses from the degassing tank, such as CO₂;
wherein one or more of the following criteria applies;
(a) wherein the first outlet and/or the first leg of the loop-part is provided with an anti-vortex device to avoid flooding of the circulation pump;
(b) wherein a ratio between a volume (internal volume determined as m³) of the degassing-tank relative to a volume (internal volume determined as m³) of the loop-part is in the range of 0.25:1 to 20:1; e.g. in the range of 0.5:1 to 15:1; such as in the range of 1:1 to 12:1; e.g. in the range of 2:1 to 10:1; such as in the range of 3:1 to 8:1; e.g. in the range of 4:1 to 6:1;
(c) wherein a numerical ratio between a volume (internal volume, determined as m³) of the degassing-tank relative to a cross section area of the first leg and/or the cross section area (determined as m²) of second leg is in the range of 10:1 to 250:1; such as in the range of 20:1 to 200:1, e.g. in the range of 30:1 to 150:1, such as in the range of 40:1 to 100:1, e.g. about 50:1;
(d) wherein a cross section area of the first leg (determined as m²) is 10% or more larger than a cross section area of the second leg (determined as m²), such as 20% larger or more; e.g. 30% larger or more, such as 40% larger or more; e.g. 50% larger or more, such as 60% larger or more; e.g. 70% larger or more, such as 80% larger or more; e.g. 90% larger or more, such as 100% larger or more; e.g. 125% larger or more, such as 150% larger or more; e.g. 200% larger or more.
(e) wherein a cross section area of the second leg (determined as m²) is 10% or more larger than a cross section area of the first leg (determined as m²), such as 20% larger or more; e.g. 30% larger or more, such as 40% larger or more; e.g. 50% larger or more, such as 60% larger or more; e.g. 70% larger or more, such as 80% larger or more; e.g. 90% larger or more, such as 100% larger or more; e.g. 125% larger or more, such as 150% larger or more; e.g. 200% larger or more; or
any combination of criteria (a) to (e).

2. The fermentation reactor according to claim 1, wherein criteria (a) applies in combination with at least one of criteria (b) (e).

3. The fermentation reactor according to claim 1, wherein the fermentation reactor comprises an ion sensor or analyser for determining the content of one or more ion species in a fermentation liquid, preferably, the one or more ion species is selected from phosphate, calcium, hydrogen, nitrate, and/or ammonium.

4. The fermentation reactor according to claim 1, wherein the circulation pump is placed in the upper half part of the first leg.

5. The fermentation reactor according to claim 1, wherein upstream from the first inlet and in the upper half of the second leg a flow reducing device is inserted.

6. The fermentation reactor according to claim 1, wherein the one or more active devices for distributing gas in the fermentation liquid is a micro- or nano-sparger for introducing and/or distributing gas into the fermentation liquid; and/or a dynamic motion device, such as a dynamic mixer.

7. The fermentation reactor according to claim 1, wherein the one or more gas inlet; the one or more water inlet; the one or more fermentation medium inlet is controlled to a computer based on the data obtained from the one or more sensors or analysers.

8. A fermentation process for production of biomass by cultivating a organism, the method comprising:
(1) supplying a fermentation medium to the fermentation reactor;
(2) adding the organism to a fermentation reactor according to claim 1;
(3) allowing the organism present in the fermentation medium to ferment, providing a fermentation liquid; and
(4) recovering the fermentation liquid from the fermentation reactor providing the biomass.

9. The fermentation process according to claim 9, wherein the organism is at least one methanotrophic organism, or a combination of organisms comprising at least one methanotrophic organism.

10. The fermentation process according to claim 9, wherein the pressure inside the loop-part and in the bottom part of the loop-part is in the range of 0-6 bar g, such as in the range of 1-5.5 bar g, e.g. in the range of 2-5 bar g, such as in the range of 2.5-4.5 bar g, e.g. in the range of 3-4 bar g.
